(19) United States
(12) Patent Application Publication
(10) Pub. No.: US 2025/0288514 A1
(43) Pub. Date: Sep. 18, 2025

(54) SKIN-SOOTHING COMPOSITION

(71) Applicant: Amorepacific Corporation, Seoul (KR)

(72) Inventors: Euidong SON, Yongin-si (KR); Insup KIL, Yongin-si (KR); Hyung Su KIM, Yongin-si (KR); Young Gyu KANG, Yongin-si (KR); Jaeyoung KO, Yongin-si (KR); Hyuk KIM, Yongin-si (KR); Chan Su RHA, Yongin-si (KR); Hyoung June KIM, Yongin-si (KR); Saitbyul PARK, Yongin-si (KR); Wonseok PARK, Yongin-si (KR)

(73) Assignee: Amorepacific Corporation, Seoul (KR)

(21) Appl. No.: 19/077,249

(22) Filed: Mar. 12, 2025

(30) Foreign Application Priority Data
Mar. 15, 2024 (KR) 10-2024-0036786

(51) Int. Cl. A61K 8/9789; A61K 8/49; A61Q 19/00

(52) U.S. Cl. CPC A61K 8/9789; A61K 8/498; A61Q 19/005; A61Q 19/007; A61K 2800/72; A61K 2800/75

(57) ABSTRACT

One aspect of the present disclosure provides a skin-soothing composition including epicatechin or a soybean extract including epicatechin, as an active ingredient.

### CROSS-REFERENCE TO RELATED APPLICATION

This application claims priority to and the benefit of Korean Patent Application No. 10-2024-0036786 filed in the Korean Intellectual Property Office on Mar. 15, 2024, the entire contents of which are incorporated herein by reference.

### BACKGROUND

### (a) Field

One aspect of the present disclosure relates to a skin-soothing composition and a cosmetic composition including the same.

### (b) Description of the Related Art

Sensitive skin reacts more easily to external stimuli than normal skin and thus easily develops symptoms of irritation, erythema, and itchiness, wherein the sensitive skin is reported to a higher transepidermal water loss (TEWL) and secrete more cytokine than the normal skin, although there is no much difference in a moisture content, pH, and melanin therebetween.

Although the above symptoms that occur in the sensitive skin are diverse, the symptoms occurring in the sensitive skin are mainly alleviated in a method of replenishing lipids in the stratum corneum of the skin mainly with ceramide to prevent the transepidermal water loss.

On the other hand, examples of the sensitive skin may be atopic dermatitis or eczema, and the atopic dermatitis or the eczema exhibits representative symptoms such as redness and itchiness. The itching symptom may occur due to losses of moisturizing factors such as filaggrin or ceramide caused by damage to the skin barrier or due to activation of nerve fibers triggered by histamine release resulting from degranulation of mast cells by various external stimuli. Accordingly, the itching symptom of the skin may be alleviated by supplementing the moisturizing factors to the skin to strengthen the skin barrier, or by inhibiting the mast cell degranulation may be to suppress the activation of nerve fibers, but conventionally, the itching relief was induced by adopting the method of supplementing the moisturizing factors to the skin to strengthen the skin barrier.

### SUMMARY

An embodiment provides a composition capable of inhibiting degranulation of mast cells and increasing claudin-1 protein expression.

Another embodiment provides a cosmetic composition that can soothe the skin by relieving itching of the skin and strengthening the skin barrier.

According to an embodiment, a skin-soothing composition includes epicatechin or a soybean extract including the same as an active ingredient.

The soybean extract may be an extract of a new variety of soybean having the deposit number KCTC14220BP.

The soybean extract may include 2 wt % to 7 wt % of epicatechin based on the total weight of an 80 wt % ethanol aqueous solution extract of the new variety of soybean.

The epicatechin or the soybean extract including the epicatechin may be included in an amount of 1 ppm to 5,000 ppm based on a total weight of the composition.

The skin soothing may be due to relief or suppression of itching.

The skin soothing may be due to improvement of the skin barrier.

The skin soothing may be due to maintaining skin moisture.

The skin soothing may be due to suppression of redness.

The skin soothing may be due to improved skin cohesion.

The skin soothing may be due to improvement in skin allergies.

According to another embodiment, a cosmetic composition including the skin-soothing composition is provided.

The cosmetic composition may include 0.1 wt % to 10 wt % of the skin-soothing composition based on a total weight of the cosmetic composition.

The cosmetic composition may be a cosmetic composition for alleviating itching.

The itching relief may be alleviating itching by inhibiting degranulation of mast cells.

According to another embodiment, a method for treating skin itching includes applying the cosmetic composition to the skin of an individual suffering from itching.

The composition according to an embodiment can inhibit degranulation of mast cells and increase claudin-1 protein expression, and therefore, a cosmetic composition according to another embodiment including the same can alleviate itching of the skin and strengthen the skin barrier.

### BRIEF DESCRIPTION OF THE DRAWINGS

FIG. 1 is a graph showing the results of the evaluation of inhibition of mast cell degranulation induction.

FIG. 2 is a photograph of artificial skin based on the results of skin cohesion evaluation.

FIG. 3 is a photograph of artificial skin showing the results of moisturizing factor evaluation.

FIG. 4 is a graph showing the results of skin redness evaluation.

### DETAILED DESCRIPTION OF THE EMBODIMENTS

Hereinafter, example embodiments of the present invention will be described in detail. However, these example embodiments are only examples and do not limit the present invention. However, this disclosure may be embodied in many different forms and is not construed as limited to the example embodiments set forth herein.

In this specification, “active ingredient” indicates an ingredient that exhibits the desired activity alone or can exhibit the activity together with a carrier that is inactive on its own.

An embodiment provides a skin-soothing composition including epicatechin or a soybean extract including the same as an active ingredient.

Mast cells are immune cells that mainly cause allergies and have a surface factor which binds to an IgE antibody on the surface. If the IgE antibody binds to the surface of the mast cells, the mast cells are activated, and the activated mast cells degranulate neurotransmitters such as histamine and the like and newly synthesize and secrete lipid mediators such as cytokines, prostaglandins, and leukotrienes, which may cause itching symptoms. In addition, inflammatory factors, which are secreted during the degranulation of the mast cells, may weaken intercellular bonding of a granular layer, which leads to skin bonding, so the skin may be easily damaged by external stimuli.

The present Inventors have confirmed that a composition containing epicatechin or a soybean extract including the same according to an embodiment may relieve itching of the skin and soothe the skin by suppressing the mast cell degranulation to inhibit the histamine release and suppress the activation of nerve fibers, wherein the composition exhibits a similar effect to ketotifen, which is conventionally used as an anti-itch agent. In addition, the composition has been confirmed to be effective in improving skin barriers, maintaining skin moisture, suppressing skin redness, enhancing skin cohesion, and improving skin allergies by suppressing the mast cell degranulation and thereby, ultimately soothe the skin.

Herein, the itching relief effect refers to an itching relief effect exerted not by an anti-inflammatory action but by inhibiting the mast cell degranulation. In general, anti-inflammation refers to actions for preventing or improving inflammations, which may occur in tissues of an organism including the skin. The inflammation includes inflammation occurring due to ultraviolet rays or oxidizing agents in the tissues including the skin, wherein the tissues may be skin tissues or tissues other than the skin, for example, lung tissues and the like. The inflammation itself may be a disease. For example, there are atopic dermatitis, chronic obstructive pulmonary disease (COPD), and the like. However, the composition according to an embodiment is unrelated to the anti-inflammatory action of preventing or improving the inflammation. In other words, the composition of an embodiment rather than manages inflammation caused by external stress but alleviates or soothes the itchiness by inhibiting the mast cell degranulation in the dry skin with a congenitally weak skin barrier where redness, tightness, itchiness, etc. frequently occur regardless of the external stress, which is completely different from conventional anti-itch compositions based on anti-inflammatory reactions. Moreover, because the conventional anti-itch compositions based on anti-inflammatory reactions, which are unrelated to the mast cell degranulation inhibition, the composition of an embodiment has a completely different composition and mechanism therefrom. (Most of the conventional anti-itch compositions maximize anti-inflammatory effects by suppressing genes such as PGE2, PLA2, COX-2, IL-2, IL-3, IL-4, IL-13, TSLP, and the like to relieve itching.) In addition, Ketotifen, a drug used as an anti-itch medicine has an effect of selectively inhibiting the histamine release through the degranulation inhibition and thus reduce itching through the inhibition of the histamine release caused by external or internal stimuli.

In addition, the composition including epicatechin or a soybean extract including the same was confirmed to relieve itching of the skin and soothe the skin by increasing expression of claudin-1 protein involved in tight junctions and strengthening the skin with weakened cell bonding.

The epicatechin included in the composition may be represented by Chemical Formula 1, but is not limited thereto:

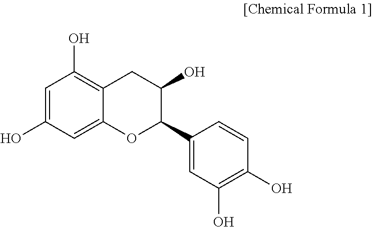 [SMILES: Oc1cc(O)c2c(c1)O[C@H](c1ccc(O)c(O)c1)[C@H](O)C2]

The soybean extract may be obtained by extracting a new variety of soybean having the deposit number KCTC14220BP, and the new soybean variety can be obtained by crossbreeding IT109098 (Napttegi soybean) as a parent plant and the semi-wild variety K7-2 as a male plant.

The new variety of soybean extract may include epicatechin in an amount of 2 wt % or more, 2.1 wt % or more, 2.2 wt % or more, 2.3 wt % or more, 2.4 wt % or more, 2.5 wt % or more, 2.6 wt % or more, 2.7 wt % or more, 2.8 wt % or more, 2.9 wt % or more, 3 wt % or more, 3.1 wt % or more, 3.2 wt % or more, 3.3 wt % or more, 3.4 wt % or more, 3.5 wt % or more, 3.6 wt % or more, 3.7 wt % or more, 3.8 wt % or more, 3.9 wt % or more, 4 wt % or more, 4.1 wt % or more, or 4.2 wt % or more, based on the total weight of the extract obtained by extracting the new variety of soybean with an 80% ethanol aqueous solution. Additionally, it may be 7 wt % or less, 6.8 wt % or less, 6.6 wt % or less, 6.4 wt % or less, 6.2 wt % or less, 6 wt % or less, 5.8 wt % or less, 5.6 wt % or less, or 5.4 wt % or less. Since the epicatechin in the soybean extract is included in the above range, the itching relief effect of the composition including it can be maximized.

The skin-soothing composition may include 1 ppm to 1,000 ppm of the epicatechin or the soybean extract including the same, for example, 1 ppm to 900 ppm, for example, 1 ppm to 800 ppm, for example, 1 ppm to 700 ppm, for example, 1 ppm to 600 ppm, for example, 1 ppm to 500 ppm, for example, 2 ppm to 1,000 ppm, for example, 3 ppm to 1,000 ppm, for example, 4 ppm to 1,000 ppm, for example, 5 ppm to 1,000 ppm, for example, 10 ppm to 1,000 ppm, for example, 10 ppm to 500 ppm based on the total weight of the composition, but is not limited thereto. By including the epicatechin or the soybean extract including the same in the composition in the above range, the itching relief effect of the composition including the same may be maximized.

The skin-soothing composition may soothe the skin in various aspects. For example, it can soothe the skin by alleviating or suppressing itching, and it can soothe the skin by maintaining the skin's moisture content. It can also soothe the skin by suppressing redness of the skin, improving the cohesion of skin cells or the skin barrier, or improving the occurrence of skin allergies.

Another embodiment provides a cosmetic composition including the composition for inhibiting degranulation of mast cells as described above.

The cosmetic composition may include the composition for inhibiting degranulation of mast cells in an amount of 0.1 wt % to 10 wt %, for example 0.1 wt % to 10 wt %, for example 0.1 wt % to 9 wt %, for example 0.1 wt % to 8 wt %, for example 0.1 wt % to 7 wt %, for example 0.1 wt % to 6 wt % for example 0.1 wt % to 5 wt %, for example 0.2 wt % to 10 wt %, for example 0.3 wt % to 10 wt %, for example 0.4 wt % to 10 wt %, for example 0.5 wt % to 10 wt %, for example 0.6 wt % to 10 wt %, for example 0.7 wt % to 10 wt %, for example 0.8 wt % to 10 wt %, for example 0.9 wt % to 10 wt %, for example 1.0 wt % to 10 wt % based on the total weight, but is not limited thereto. Since the composition for inhibiting degranulation of mast cells is included in the cosmetic composition in the above content range, the cosmetic composition can be used without causing any other side effects on the skin, and the itching relief effect can be maximized.

The cosmetic composition can alleviate itching of the skin. The itching of the skin may be itchiness that occurs due to the loss of moisturizing factors in the skin caused by a dry external environment, or itchiness that occurs due to degranulation of mast cells. When the cosmetic composition is used on the skin, it can suppress the degranulation of mast cells, inhibit histamine release, inhibit nerve fiber activation, and relieve itching of the skin. In addition, it can suppress skin redness and inhibit the occurrence of skin allergies, thereby soothing the skin. In addition, the cosmetic composition can alleviate itching of the skin by strengthening the cell adhesion (tight junction) of the granular layer in the skin epidermis, that is, strengthening the cell cohesion and improving the skin barrier. In addition, it can suppress the loss of moisturizing factors in the skin by strengthening skin cell cohesion, maintain skin moisture, and relieve and soothe itching of the skin. Since the cosmetic composition is basically applied to the skin, it can be manufactured in any formulation that is commonly manufactured with reference to cosmetic compositions in the art. For example, it can be formulated as a solution, suspension, emulsion, paste, gel, cream, lotion, powder, soap, surfactant-containing cleansing, oil, powder foundation, emulsion foundation, emulsion obtained by dispersing oil phase in water phase, emulsion obtained by dispersing water phase in oil phase, microneedle, foam, aerosol, wax foundation, and spray, but is not limited thereto.

The cosmetic composition may have formulations such as, but not limited to, a softening toner, an astringent toner, a nourishing toner, a nourishing cream, a massage cream, an eye cream, an eye essence, an essence, a cleansing cream, a cleansing lotion, a cleansing foam, a cleansing water, a pack, a powder, a body lotion, a body cream, a body essence, a body cleanser, a hair dye, a shampoo, a rinse, a hair conditioner, a topical agent, an ointment, a gel, a cream, a patch, a spray, a powder, and a skin adhesive type.

In addition to the essential ingredients mentioned above, in the above formulation, other ingredients can be appropriately selected and mixed without difficulty by those skilled in the art depending on the type of external preparation or intended use.

When the chemical formulation of the cosmetic composition is a paste, cream, or gel, the carrier component may include animal oil, vegetable oil, wax, paraffin, starch, tragacanth, cellulose derivatives, polyethylene glycol, silicone, bentonite, silica, talc, zinc oxide, or the like.

If the chemical formulation of the cosmetic composition is a powder or spray, the carrier component may include lactose, talc, silica, aluminum hydroxide, calcium silicate, polyamide powder, etc., and especially in the case of a spray, the composition may additionally include a propellant such as chlorofluorohydrocarbon, propane/butane, dimethyl ether, etc.

When the chemical formulation of the cosmetic composition is a solution or emulsion, the carrier component may include a solvent, a solubilizer, an emulsifier, etc., and specifically, water, ethanol, isopropanol, ethyl carbonate, ethyl acetate, benzyl alcohol, benzyl benzoate, propylene glycol, 1,3-butyl glycol oil, glycerol aliphatic ester, polyethylene glycol, fatty acid ester of sorbitan, etc.

When the chemical formulation of the cosmetic composition is a suspension, the carrier component may include a liquid diluent such as water, ethanol, or propylene glycol; a suspending agent such as ethoxylated isostearyl alcohol, polyoxyethylene sorbitol ester, or polyoxyethylene sorbitan ester; microcrystalline cellulose, aluminum metahydroxide, bentonite, agar, or tragacanth.

When the chemical formulation of the cosmetic composition is a surfactant-containing cleansing, the carrier component may include an aliphatic alcohol sulfate, an aliphatic alcohol ether sulfate, a sulfosuccinic acid monoester, an isethionate, an imidazolinium derivative, methyl taurate, a sarcosinate, a fatty acid amide ether sulfate, an alkyl amidobetaine, a fatty alcohol, a fatty acid glyceride, a fatty acid diethanolamide, a vegetable oil, a lanolin derivative, an ethoxylated glycerol fatty acid ester, and the like.

Meanwhile, another embodiment provides a method for treating skin itching, which includes applying the cosmetic composition to the skin of an individual suffering from itching.

The applying of the cosmetic composition to the skin of the subject causing the itching may be applied without limitation in any form in which the cosmetic composition can be applied and exhibit an effect, such as by application or injection. In addition, the method for treating skin itching refers to a method of reducing skin itching symptoms, such as alleviating, suppressing, and reducing skin itching.

Hereinafter, the present disclosure is illustrated in more detail with reference to examples. However, these examples are exemplary, and the present disclosure is not limited thereto.

### Preparation of Soybean Extract

### Preparation Example

Soybeans with a deposit number No. KCTC14220BP, a new variety, was ground into powder by using a grounder (Tube Mill 100 Control, IKA). 100 g of the obtained soybean powder was added to 1 L of an 80% ethanol aqueous solution and extracted at room temperature (25° C.) for 24 hours, and an extract therefrom was filtered with a filter paper. Subsequently, the filtered extract was dried by using a vacuum concentrator to obtain 15.7 g of the extract.

### Preparation of Compositions

### Example 1

A skin-soothing composition was prepared by adding 1 g of epicatechin (EC, CAS NO. 490-46-0) to 100 g of purified water or dimethyl sulfoxide (DMSO) to prepare a stock and diluting the stock to 1 ppm by using a medium.

### Example 2

A skin-soothing composition was prepared by adding 1 g of epicatechin (EC, CAS NO. 490-46-0) to 100 g of purified water or dimethyl sulfoxide (DMSO) to prepare a stock and diluting the stock to 10 ppm by using a medium.

### Example 3

A skin-soothing composition was prepared by adding 1 g of epicatechin (EC, CAS NO. 490-46-0) to 100 g of purified water or dimethyl sulfoxide (DMSO) to prepare a stock and diluting the stock to 50 ppm by using a medium.

### Example 4

A skin-soothing composition was prepared by adding 1 g of the soybean extract according to the preparation example to 100 g of purified water or dimethyl sulfoxide (DMSO) to prepare a stock and diluting the stock to 1 ppm by using a medium.

### Example 5

A skin-soothing composition was prepared by adding 1 g of the soybean extract according to the preparation example to 100 g of purified water or dimethyl sulfoxide (DMSO) to prepare a stock and diluting the stock to 10 ppm by using a medium.

### Example 6

A skin-soothing composition was prepared by adding 1 g of the soybean extract according to the preparation example to 100 g of purified water or dimethyl sulfoxide (DMSO) to prepare a stock and diluting the stock to 50 ppm by using a medium.

### Example 7

A skin-soothing composition was prepared by adding 1 g of the soybean extract according to the preparation example to 100 g of purified water or dimethyl sulfoxide (DMSO) to prepare a stock and diluting the stock to 100 ppm by using a medium.

### Comparative Example 1

Purified water or dimethyl sulfoxide (DMSO) was used.

### Comparative Example 2

A composition was prepared by adding 1 g of palmitoylethanolamide (PEA) to 100 g of purified water or dimethyl sulfoxide (DMSO) to prepare a stock and diluting the stock to 1 ppm by using a medium.

### Comparative Example 3

A composition was prepared by adding 1 g of palmitoylethanolamide (PEA) to 100 g of purified water or dimethyl sulfoxide (DMSO) to prepare a stock and diluting the stock to 10 ppm by using a medium.

### Comparative Example 4

A composition was prepared by adding 1 g of palmitoylethanolamide (PEA) to 100 g of purified water or dimethyl sulfoxide (DMSO) to prepare a stock and diluting the stock to 50 ppm by using a medium. Because PEA was suspended at the 50 ppm, this composition was not used for an experiment.

### Comparative Example 5

A composition was prepared by adding 1 g of (−)-epigallocatechin gallate (EGCG) to 100 g of purified water or dimethyl sulfoxide (DMSO) to prepare a stock and diluting the stock to 1 ppm.

### Comparative Example 6

A composition was prepared by adding 1 g of (−)-epigallocatechin gallate (EGCG) to 100 g of purified water or dimethyl sulfoxide (DMSO) to prepare a stock and diluting the stock to 10 ppm.

### Comparative Example 7

A composition was prepared by adding 1 g of (−)-epigallocatechin gallate 5 (EGCG) to 100 g of purified water or dimethyl sulfoxide (DMSO) to prepare a stock and diluting the stock to 50 ppm. EGCG at 50 ppm was not used for an experiment due to cell toxicity.

Components and concentrations in the compositions according to Examples 1 to 6 and Comparative Examples 1 to 7 are summarized in Table 1.

TABLE 1

| | | Soybean | | |
|---|---|---|---|---|
| | Epicatechin | extract | PEA | EGCG |
| | (ppm) | (ppm) | (ppm) | (ppm) |
| Example 1 | 1 | — | — | — |
| Example 2 | 10 | — | — | — |
| Example 3 | 50 | — | — | — |
| Example 4 | — | 1 | — | — |
| Example 5 | — | 10 | — | — |
| Example 6 | — | 50 | — | — |
| Example 7 | — | 100 | — | — |
| Comparative Example 1 | — | — | — | — |
| Comparative Example 2 | — | — | 1 | — |
| Comparative Example 3 | — | — | 10 | — |
| Comparative Example 4 | — | — | 50 | — |
| Comparative Example 5 | — | — | — | 1 |
| Comparative Example 6 | — | — | — | 10 |
| Comparative Example 7 | — | — | — | 50 |

### Evaluation 1: Evaluation of Suppression of Degranulation Induction

### 1) Culturing of RBL-2H3 Cell Line

A RBL-2H3 cell line (basophil leukemia cell line) was obtained from the Korean cell line bank (22256) and cultured in a CO₂ incubator at 37° C. under 5% CO₂ condition. A DMEM media (w/10% FBS, 1% antibiotics) was used as a medium.

### 2) Treatment of Substances to RBL-2H3 Cell Line

The RBL-2H3 was seeded by 4×10⁴ cells/well (cells/well) in 96 wells (cell culture dishes) in the 37° C. and 5% CO₂ incubator to confluency of about 80%. Subsequently, after treating them with DNP-IgE (200 ng/ml), an allergen substance, for one day, each of the compositions according to Examples 1 to 6 and Comparative Examples 1, 2, 3, 5, and 6 were applied thereto, while activating secondary mast cells with DNP-BSA (1 μg/ml).

### 3) Measurement of Mast Cell Degranulation (β-Hexosaminidase Assay)

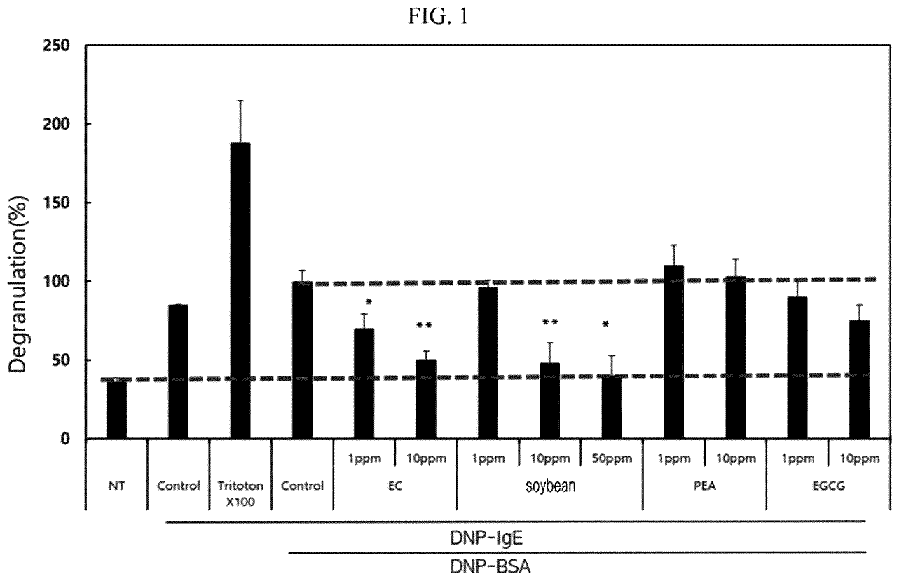

After culturing the substance-treated cells in 2) in the incubator for 1 hour, 50 μl sup from each well was transferred to new 96 well plates, and 50 μl of a substrate solution was added thereto and then, reacted for 1 hour in the incubator. After adding 100 μl of a Stop solution thereto to confirm that it turned yellow, absorbance at 405 nm was measured. Triton-X100 was used as a positive material. The results are shown in FIG. 1.

Referring to FIG. 1, the cases of applying the compositions of Examples 1 to 6, compared with the cases of applying the compositions of Comparative Examples 1 2, 3, 5, and 6, were confirmed to be significantly suppressed from the mast cell degranulation. In other words, the mast cell degranulation induced by allergens was suppressed, when treated with a composition including epicatechin or a soybean extract including the same. Accordingly, a cosmetic composition including the composition was confirmed to have an effect of alleviating or suppressing skin itching as well as an effect of suppressing skin allergies by the mast cell degranulation.

### Evaluation 2: Evaluation of Skin Cohesion and Moisturizing Factor

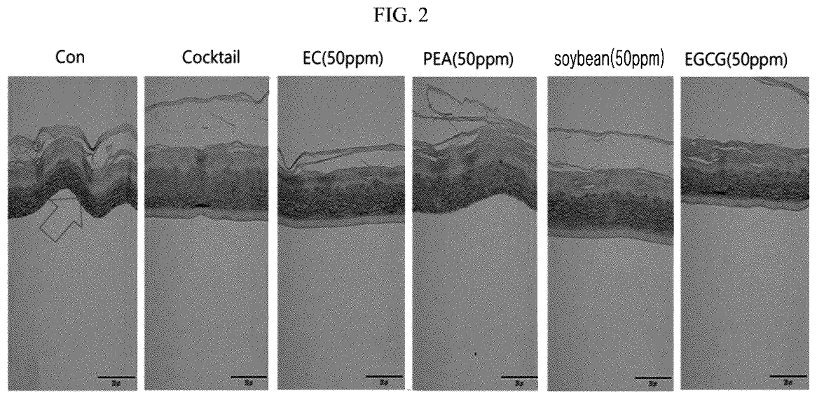

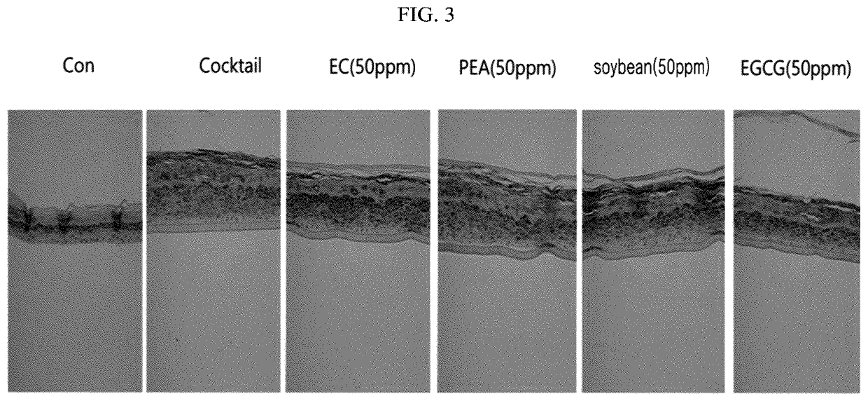

After applying an inflammation cocktail (IFN-γ (20 ng/ml)+TNF-α (20 ng/ml)) and the compositions of Comparative Examples 1, 4, and 7 and Examples 3 and 6 to artificial skin, expression levels of claudin-1 protein of a weakened skin binding factor and filaggrin protein of a moisturizing factor) were compared, and the results are shown in FIGS. 2 and 3.

Referring to FIG. 2, when the inflammation cocktail alone was applied to artificial skin, the expression level of the claudin-1 protein decreased, which confirmed that cell-cell bonding was weakened, thereby blurring boundaries between cell layers. The artificial skins, to which the compositions of Comparative Examples 1, 4, and 7 were applied, were not recovered from the decrease in the protein expression of the claudin-1 protein but still exhibited blurry boundaries between cells, but the artificial skins, to which the compositions of Examples 3 and 6 were applied, were recovered from the decrease in the protein expression of the claudin-1 protein and exhibited relatively clear boundaries between cells. In other words, the compositions of the examples containing epicatechin or a soybean extract containing the same were confirmed to be effective in increasing an amount of the protein expression of the claudin-1 protein.

Referring to FIG. 3, when the inflammation cocktail alone was applied to the artificial skin, the filaggrin protein, which is a moisturizing factor, was less expressed, resulting in blurry boundaries between cells. The artificial skins, to which the compositions of Comparative Examples 1, 4, and 7 were applied, were not recovered from the decrease of the protein expression and thus exhibited still blurry boundaries between cells, but the artificial skins, to which the compositions of Examples 3 and 6 were applied, were recovered from the decrease of the protein expression and exhibited relatively clear boundaries between cells. In other words, the compositions of the examples including epicatechin or a soybean extract including the same were confirmed to be effective in increasing an amount of the filaggrin protein expression.

On the other hand, referring to FIGS. 2 and 3, even when applying the composition of Comparative Example 4 including palmitoylethanolamide (PEA), which is known to have no effect of suppressing the mast cell degranulation but an effect of suppressing skin inflammation, the decreases in the claudin-1 and filaggrin protein expressions were not recovered.

Referring to Evaluations 2 and 3, the compositions including epicatechin or a soybean extract including the same were confirmed to improve skin moisturizing capacity and strengthen skin barriers by improving tight junctions between cells of a granular layer of the skin.

### Evaluation 3: Skin Redness Evaluation

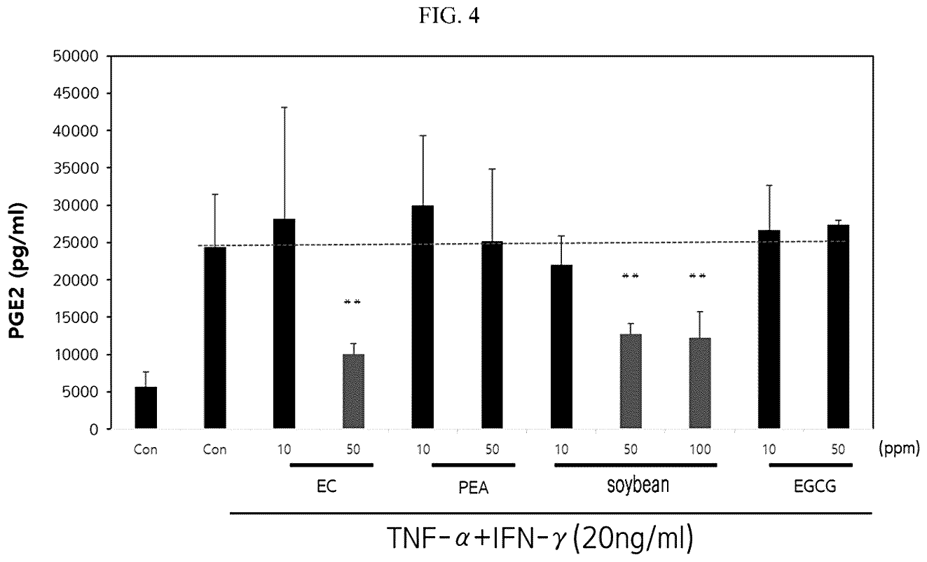

After treating the artificial skin with an inflammatory factor (TNF-α+IFN-γ (20 ng/ml)) and applying the compositions of Examples 2, 3, 5, 6, and 7 and Comparative Examples 3, 4, 6, and 7 thereto, an amount of PGE2 (Prostaglandin E2) was measured. PGE2 is a factor that induces vasodilation and causes skin redness, wherein the smaller amount of PGE2, the less vasodilation induction, thereby suppressing the skin redness, and the skin redness evaluation results are shown in FIG. 4.

Referring to FIG. 4, when the compositions of Examples 3, 6, and 7 were applied, the amount of PGE2 was suppressed. Accordingly, the composition including epicatechin or a soybean extract including the same was confirmed to suppress the skin redness.

Although the preferred embodiments of the present invention have been described in detail, the scope of the present invention is not limited thereto, and various modifications and improvements by those skilled in the art using the basic concept of the present invention defined in the following claims are also within the scope of the invention.

### DEPOSIT NUMBER

Korea Research Institute of Bioscience and Biotechnology, KCTC14220BP, Jun. 19, 2020.

1. A skin-soothing composition comprising a soybean extract including the epicatechin as an active ingredient.

2. The skin-soothing composition of claim 1, wherein the soybean extract is an extract of a new variety of soybean with a deposit number of KCTC14220BP.

3. The skin-soothing composition of claim 2, wherein the soybean extract includes 2 wt % to 7 wt % of epicatechin based on a total weight of an 80 wt % ethanol aqueous solution extract of the new variety of soybean.

4. The skin-soothing composition of claim 1, wherein the epicatechin or the soybean extract including the same is included at 1 ppm to 5,000 ppm based on a total weight of the composition.

5. The skin-soothing composition of claim 1, wherein the skin soothing is achieved by relieving or suppressing itching.

6. The skin-soothing composition of claim 1, wherein the skin soothing is achieved by improving skin barriers.

7. The skin-soothing composition of claim 1, wherein the skin soothing is achieved by skin moisturizing.

8. The skin-soothing composition of claim 1, wherein the skin soothing is achieved by suppressing skin redness.

9. The skin-soothing composition of claim 1, wherein the skin soothing is achieved by improving skin cohesion.

10. The skin-soothing composition of claim 1, wherein the skin soothing is achieved by improving skin allergies.

11. A cosmetic composition comprising the composition according to claim 1.

12. The cosmetic composition of claim 11, wherein the skin-soothing composition is included in an amount of 0.1 wt % to 10 wt % based on a total weight of the cosmetic composition.

13. The cosmetic composition of claim 11, wherein the skin-soothing composition is a cosmetic composition for alleviating itching.

14. The cosmetic composition of claim 13, wherein the itching relief is characterized to be achieved by suppressing degranulation of mast cells.

15. A treatment for skin itching, which includes applying the cosmetic composition of claim 11 to the skin of an individual suffering from itching.
